(19) United States
(12) Patent Application Publication
(10) Pub. No.: US 2026/0108321 A1
(43) Pub. Date: Apr. 23, 2026

(54) BIOPSY DEVICES AND METHODS THEREOF FOR DEPLOYING MARKERS

(71) Applicant: BARD PERIPHERAL VASCULAR, INC., Franklin Lakes, NJ (US)

(72) Inventors: Matthew CARTWRIGHT, Chandler, AZ (US); Summer L. FORD, Queen Creek, AZ (US); Genevieve MESSINA, Mesa, AZ (US); William E. PARMENTIER, Gilbert, AZ (US); Kyra TEMPLE, Mesa, AZ (US); Peng ZHENG, Chandler, AZ (US)

(73) Assignee: BARD PERIPHERAL VASCULAR, INC., Franklin Lakes, NJ (US)

(21) Appl. No.: 19/115,459

(22) PCT Filed: Sep. 29, 2022
(86) PCT No.: PCT/US2022/077248

§ 371 Date: Mar. 26, 2025

(51) Int. Cl. A61B 90/00; A61B 1/018; A61B 1/267; A61B 10/02; A61B 10/04; A61M 25/00; A61M 25/06

(52) U.S. Cl. CPC A61B 90/39; A61B 1/018; A61B 1/2676; A61B 10/0266; A61B 10/04; A61B 2090/0811; A61B 2090/3912; A61B 2090/3987; A61M 25/0082; A61M 2025/0681

(57) ABSTRACT

A device for deploying markers includes a deployment catheter, a first push rod, and a second push rod. The deployment catheter includes an elongate body and a cutting tip at a distal end of the elongate body. The deployment catheter defining a first lumen separate from a second lumen. The first lumen having a first proximal opening and a distal opening at the cutting tip. A deployment portion positioned therebetween. The second lumen having a deployment opening and a second proximal opening. The second lumen shaped and sized to hold a plurality of markers. The second push rod independently pushes each of the plurality of markers from the second lumen into the first lumen into a deployed position within the deployment portion. The first push rod pushes the one of the plurality of markers from the deployed position out of the distal opening at the cutting tip.

### TECHNICAL FIELD

The present specification generally relates to biopsy devices and, more specifically, to devices for deploying markers.

### BACKGROUND

Markers can be used to mark a site of a tissue biopsy. Current marker deployment is accomplished using a needle device through the chest wall. There are inherent risks with this procedure. The markers may be detectable by a scan or other procedure on the subject to view where the biopsy was taken without an invasive procedure. The markers may be provided with a biopsy device to remove a tissue sample and mark the location with a single tool. However, conventional biopsy devices use movement of a cutting device that removes the tissue sample from the biopsy site to deploy the markers. This automatic deployment of the markers restricts the operator from controlling deployment of the marker.

### SUMMARY

In one aspect, a device for deploying markers is provided. The device for deploying markers includes a deployment catheter, a first push rod, and a second push rod. The deployment catheter includes an elongate body and a cutting tip at a distal end of the elongate body. The deployment catheter defining a first lumen and a second lumen. The first lumen is separate from the second lumen. The first lumen has a first proximal opening and a distal opening at the cutting tip. A deployment portion is positioned therebetween. The second lumen has a deployment opening and a second proximal opening. The second lumen is shaped and sized to hold a plurality of markers. The deployment opening is open to the deployment portion of the first lumen. The first push rod is positionable within and movable along a length of the first lumen. The second push rod is positionable within and movable along a length of the second lumen independently of the first push rod. The second push rod is configured to independently push each one of the plurality of markers from the second lumen into the first lumen via the deployment opening and into a deployed position within the deployment portion. The first push rod configured to push the one of the plurality of markers from the deployed position out of the distal opening at the cutting tip.

In another aspect, a system for performing a biopsy is provided. The system includes a bronchoscope with a channel formed therein and a device. The device is configured to be inserted into the channel of the bronchoscope. The device includes a deployment catheter, a first push rod, and a second push rod. The deployment catheter includes an elongate body and a cutting tip at a distal end of the elongate body. The elongate body defines a first lumen and a second lumen. The first lumen is separate from a second lumen. The first lumen has a first proximate opening and a distal opening at the cutting tip. A deployment portion is positioned therebetween. The second lumen has a deployment opening and a second proximate opening. The second lumen is shaped and sized to hold a plurality of markers. The deployment opening is open to the deployment portion of the first lumen. The first push rod is positionable within and movable along a length of the first lumen. The second push rod is positionable within and movable along a length of the second lumen independently of the first push rod. The second push rod is configured to independently push each one of the plurality of markers from the second lumen into the first lumen via the deployment opening and into a deployed position within the deployment portion. The first push rod is configured to push the one of the plurality of markers from the deployed position out of the distal opening at the cutting tip.

In yet another aspect, a method of marking a biopsy site is provided. The method includes inserting a bronchoscope into an airway of a subject, positioning the bronchoscope at a desired target marking site, retracting a first push rod along a first lumen, via a first handle of a device for deploying markers inserted into the bronchoscope via an instrument port, to move the first push rod into a loading position, advancing a second push rod along a second lumen of the device for deploying markers, via a second handle of the device for deploying markers inserted into the bronchoscope. The advancing of the second push rod moves at least one marker of a plurality of markers stored in the second lumen into the first lumen via a ramped portion coupling the second lumen to the first lumen, and advancing the first push rod along the first lumen from the loading position into a deployment position via the first handle of the device for deploying markers such that a top portion of the first push rod makes contact with the at least one marker of the plurality of markers to advance the at least one marker of the plurality of markers through a deployment portion and exits the device for deploying markers via a deployment opening.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts an environmental side view of a subject and an illustrative biopsy device for performing a biopsy according to one or more embodiments shown and described herein;

FIG. 2A schematically depicts an isolated side view of the biopsy device of FIG. 1 having a plurality of illustrative markers loaded within the biopsy device, according to one or more embodiments shown and described herein;

FIG. 2B schematically depicts a perspective view of an illustrative cutting tip of the biopsy device of FIG. 2A, according to one or more embodiments shown and described herein;

FIG. 3A schematically depicts an isolated side view of the biopsy device of FIG. 1 without a marker loaded within the biopsy device, according to one or more embodiments shown and described herein;

FIG. 3B schematically depicts a perspective view of a cutting tip of the biopsy device of FIG. 3A, according to one or more embodiments shown and described herein;

FIG. 4 schematically depicts a cross-sectional side view of the biopsy device of FIG. 2B, illustrating a partial view of an illustrative first push rod and an illustrative second push rod, according to one or more embodiments shown and described herein;

FIG. 5A schematically depicts a partial cross-sectional view of the biopsy device of FIG. 2B with the first push rod in an extended position and the second push rod in a retracted position with a marker positioned in a second lumen according to one or more embodiments shown and described herein;

FIG. 5B schematically depict a partial cross-sectional view of the biopsy device of FIG. 5A with the first push rod in a retracted position and the second push rod in a deployed position forcing the marker into a first lumen according to one or more embodiments shown and described herein;

FIG. 5C schematically depicts a partial cross-sectional view of the biopsy device of FIG. 5B with the first push rod in the extended position to deploy the marker from the first lumen and the second push rod in a retracted position according to one or more embodiments shown and described herein;

FIG. 6 schematically depicts a cross-sectional side view of another illustrative biopsy device, according to one or more embodiments shown and described herein; and

FIG. 7 schematically depicts a flowchart of an illustrative method of operating the biopsy device of FIGS. 1 and 6, according to one or more embodiments shown and described herein.

### DETAILED DESCRIPTION

Embodiments described herein are directed to biopsy devices that include a bronchoscope device with a lumen for deploying a plurality of markers in lung biopsy site of a subject via the lumen of the bronchoscope device. The markers may be a radiopaque fiducial marker and the biopsy device is capable of deploying several markers without the need to reload after each marker deployment. As such, conventional devices are limited to a one-time deployment and needs to be reloaded after every deployment.

The biopsy devices disclosed herein generally include a deployment catheter defining a pair of lumens formed therein, a cutting tip formed at a distal end of the an elongate body of the deployment catheter, and a pair of push rods disposed in the pair of lumens. A first push rod is positionable within and movable along a length of the first lumen and the second push rod is positionable within and movable along a length of the second lumen independently of the first push rod. A plurality of markers are disposed in one of the pair of lumens. A marker advance portion of the deployment catheter connects the pair of lumens such that at least one of the plurality of markers may be advanced from one lumen to the other via direct contact when moving the second push rod from a retracted position to an extended position. Once the at least one marker is positioned into a deployment portion of the other lumen, the first push rod is moved such that direct contact is made with the at least one marker to force the at least one marker out of the other lumen and into a deployed position.

The biopsy devices disclosed herein may repeat the movement of pushing the first push rod and the second push rod to continuously and repeatedly deploy the plurality of markers stored in the lumens of the deployment catheter. As such, the ability to not need to reload for each deployment is an improvement over conventional devices. The biopsy devices disclosed herein further provide improvements over conventional devices by permitting the continuous and repeatedly deploying of the plurality of markers, which in turn provide direct localization of previous biopsy, surgery sites, or suspicious areas for disease monitoring and/or subsequent procedures. Further, the devices disclosed herein reduce the risk of pneumothorax due to a lung puncture because the biopsy devices described herein permit the tissue sample to be gathered using a transbronchial procedure, which uses existing pathways of the lung itself to gather the tissue from an internal position as opposed to conventional techniques that puncture through the lung from an external position to gather a sample.

Various embodiments of the systems and methods of the biopsy device will be described in more detail herein.

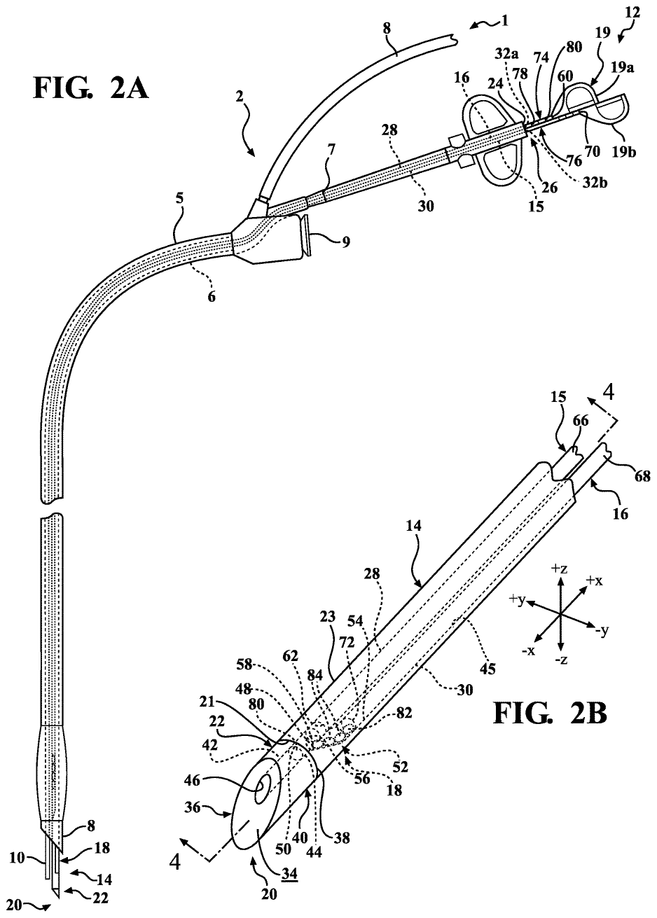

As used herein, the term “device longitudinal direction” refers to the forward-rearward direction of the device (i.e., in a +/−X direction of the coordinate axes depicted in FIG. 2B). The term “device lateral direction” refers to the cross-direction (i.e., along the Y axis of the coordinate axes depicted in FIG. 2B), and is transverse to the longitudinal direction. The term “device vertical direction” refers to the upward-downward direction of the device (i.e., in the +/−Z direction of the coordinate axes depicted in FIG. 2B). As used herein, “upper” or “uppermost” is defined as generally being towards the positive Z direction of the coordinate axes shown in the drawings and “lower”, “lowermost” or “below” is defined as generally being towards the negative Z direction of the coordinate axes shown in the drawings.

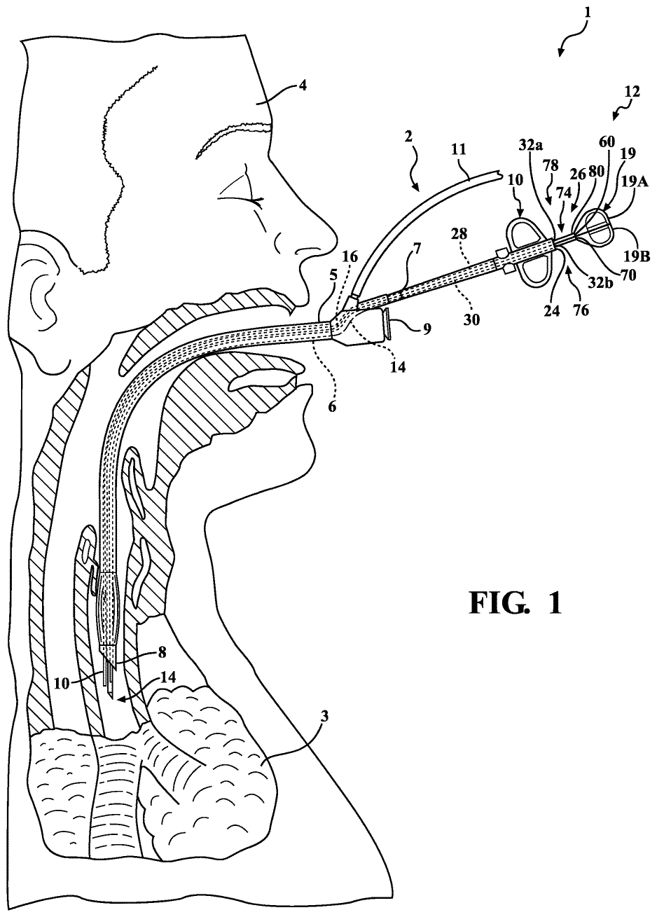

Referring now to the figures, FIG. 1 generally depicts a first aspect of an assembly 1 for performing a biopsy. The assembly 1 may include a bronchoscope 2 and a combination biopsy and marker deployment device 12 that is positioned within the bronchoscope 2, as discussed in greater detail herein. The bronchoscope 2 may be a conventional bronchoscope for viewing airways in a pair of lungs 3 of a subject 4. The bronchoscope 2 may include an elongated tube 5 that has a channel 6 formed therein and extending between an instrument port 7 of the bronchoscope 2 and a tip portion 8, or deployment catheter, of the bronchoscope 2. The instrument port 7 may be in communication with the channel 6 of the elongated tube 5 such that the combination biopsy and marker deployment device 12 may be inserted into the channel 6 of the bronchoscope 2, as discussed in greater detail herein. The bronchoscope 2 may further include a camera 9 or other lens and a flexible elongated member 10 that extends beyond the tip portion 8 of the bronchoscope 2. The flexible elongated member 10 may include a fiber-optic system to work in conjunction with the camera 9 to capture images and/or video (e.g., a sequence of images).

The flexible elongated member 10 may be communicatively coupled to the camera 9 or other lens such that a user may use the camera 9 or lens with the flexible elongated member 10 to view various anatomies of the subject 4, such as the airway and lungs 3. A second elongated member 11 may be coupled to the bronchoscope 2 to provide either ambient or enhanced light into the channel 6 and/or into the airway of the subject 4 such that the fiber-optic system of the flexible elongated member 10 and the camera 9 or lens may be able to see the various anatomy of the subject 4.

The instrument port 7 may be in communication with the channel 6 of the elongated tube 5 such that portions of the combination biopsy and marker deployment device 12 may be inserted into the channel 6 via the instrument port 7. While the assembly 1 is depicted to include the bronchoscope 2, the assembly 1 may include other medical devices for use in conjunction with the combination biopsy and marker deployment device 12, such as, for example, an endoscope, a cystoscope, and/or the like. As discussed in greater detail herein, the combination biopsy and marker deployment device 12 may be used in conjunction with the bronchoscope 2 to retrieve a tissue sample at a biopsy site of the subject 4 while viewing the biopsy site with the bronchoscope 2. The combination biopsy and marker deployment device 12 may additionally or alternatively be used to mark the location of the biopsy site, as discussed in greater detail herein. Further, while the bronchoscope 2 is depicted in FIG. 1 as being inserted into the airway of the subject via an oral cavity, this is non-limiting and the bronchoscope 2 may be inserted via the nasal passageway.

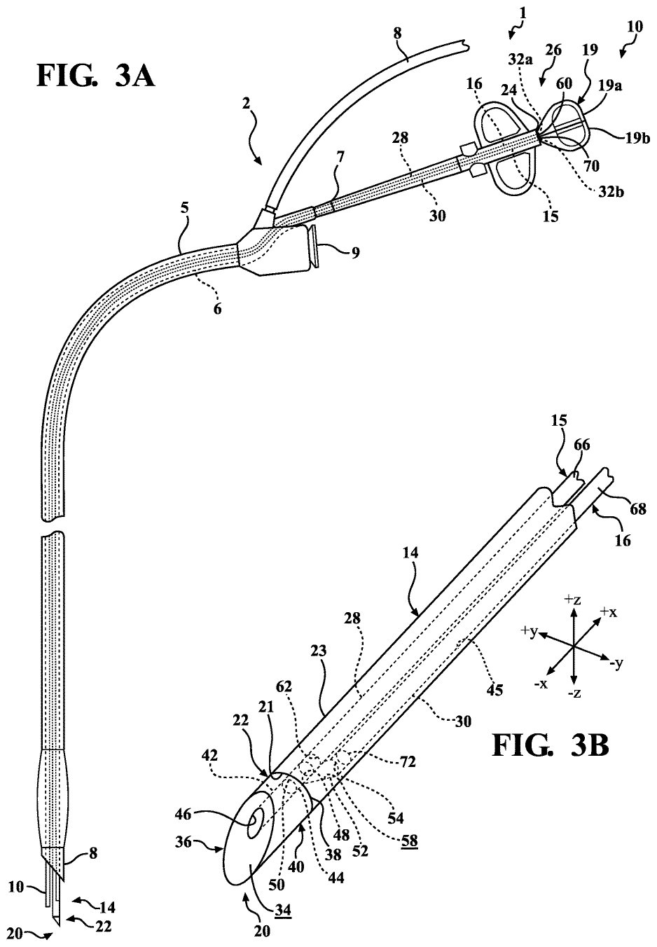

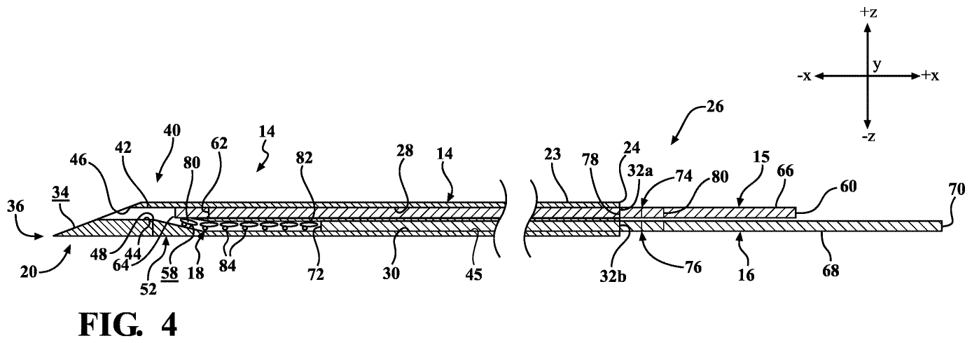

Still referring to FIG. 1 and now also referring to FIGS. 2A-2B, 3A-3B and 4, the combination biopsy and marker deployment device 12 may include a deployment catheter 14, an actuator 19 and a cutting tip 20. A first push rod 15 (FIGS. 2B, 3B and 4), a second push rod 16 (FIGS. 2B, 3B and 4) and a plurality of markers 18 (FIGS. 2B and 4) are positioned within an elongate body 23 of the deployment catheter 14, as discussed in greater detail herein. The deployment catheter 14 includes a coupling cutting portion 21 at a distal end 22 and an opening 24 at a proximal end 26 opposite the distal end 22. A pair of lumens are separated from one another, such as a first lumen 28 and a second lumen 30, and extend within the deployment catheter 14 and each of the first lumen 28 and the second lumen 30 open at a first lumen proximate opening 32a and a second lumen proximate opening 32b at the opening 24 respectively, as discussed in greater detail herein. In some embodiments, the first lumen 28 and the second lumen 30 are spaced apart to create the separation. In other embodiments, the first lumen 28 and the second lumen 30 are adjacent to one another and may be separated by a common wall or other structures.

The opening 24 permits objects to be inserted into the deployment catheter 14. For example, the opening 24 provides access to the first lumen proximate opening 32a and the second lumen proximate opening 32b, which in turn provides access to the first lumen 28 and the second lumen 30. The deployment catheter 14 is configured to extend through the instrument port 7 and into the channel 6. In some embodiments, the deployment catheter 14 may extend beyond the tip portion 8 of the bronchoscope 2 when fully inserted. In other embodiments, the deployment catheter 14 may remain housed within the tip portion 8 of the bronchoscope 2.

In some embodiments, a diameter of the proximal end 26 and a diameter of the distal end 22 of the deployment catheter 14 are equal or substantially equal. In other embodiments, the diameter of the proximal end 26 of the deployment catheter 14 may be tapered, or may taper to different a diameter along a length. In other embodiments, the diameter of the distal end 22 of the deployment catheter 14 may be tapered, or may taper to a different diameter along a length. In other embodiments, the diameter of the proximal end 26 of the deployment catheter 14 may be larger than the diameter of the distal end 22 of the deployment catheter 14. In other embodiments, the diameter of the distal end 22 of the deployment catheter 14 may be larger than the diameter of the proximal end 26 of the deployment catheter 14.

In some embodiments, the deployment catheter 14 maintains a uniform shape between the proximal end 26 and the distal end 22. In other embodiments, the deployment catheter 14 may include non-uniform shapes, such as tapers or narrowing of various portions of the deployment catheter 14 between the proximal end 26 and the distal end 22.

The cutting tip 20 may include a beveled surface 34 at a distal end 36 and a coupling portion 38 at a proximal end 40. A deployment portion 42 extends in a device longitudinal direction (i.e., in the +/−X direction depicted in FIG. 2B) between the beveled surface 34 and the coupling portion 38. The deployment portion 42 may be a lumen and may have a marker receiving opening 44 at the coupling portion 38 and an opposite marker deployment opening 46 at the beveled surface 34. In some embodiments, the cutting tip 20 may be removably coupled to the distal end 22 of the deployment catheter 14 via the coupling portion 38. In such embodiments, the coupling portion 38 of the cutting tip 20 may be press fit into the distal end 22 of the deployment catheter 14 such that the coupling cutting portion 21 is coupled to the coupling portion 38 of the cutting tip 20. In other embodiments, the cutting tip 20 may be integrally formed with the deployment catheter 14, and the deployment catheter 14 may be provided without coupling cutting portion 21.

In other embodiments, the coupling portion 38 of the cutting tip 20 may be coupled to the coupling cutting portion 21 of the distal end 22 of the deployment catheter 14 via a fastener, such as a bolt and nut, hook and loop fastener, setscrew, screws, adhesive, epoxy, and/or the like. As discussed in greater detail herein, the cutting tip 20 removably coupled to the distal end 22 of the deployment catheter 14 may permit for the plurality of markers 18 to be loaded into the deployment catheter 14 at the distal end 22, as discussed herein.

In some embodiments, the beveled surface 34 of the cutting tip 20 may be used for sample acquisition by removing and storing a tissue sample from a biopsy site. In some embodiments, the sample may be stored in the marker deployment opening 46 of the deployment portion 42. In other embodiments, the collected sample may be stored against the beveled surface 34 or other parts of the cutting tip 20. In some embodiments, the cutting tip 20 may be moved into a desired sample area to cut and retrieve the tissue sample. In some embodiments, the cutting tip 20 may be actuated via another mechanical or electrical device to cut and retrieve the tissue sample. In some embodiments, a vacuum may be attached to suck the sample or to retain the sample against the beveled surface 34. For example, a vacuum may be coupled to the deployment catheter 14 such as to couple the vacuum to the first lumen 28. The vacuum may be coupled or attached to the first lumen in place of the actuator 19 (a first handle 19a and a second handle 19b). That is, the vacuum may be coupled to the first lumen 28 external to the bronchoscope 2.

Still referring to FIGS. 1-4, the first lumen 28 may extend a length of the deployment catheter 14 between the opening 24 and the cutting tip 20. The second lumen 30 extends generally along (e.g., generally parallel) to the first lumen 28 between the opening 24 and a deployment opening 48 of the second lumen 30, as discussed in greater detail herein. In some embodiments, the second lumen 30 extends within the deployment catheter 14 a length less than a length that the first lumen 28 extends within the deployment catheter 14. In some embodiments, the first lumen 28 may be positioned above the second lumen 30 in the vertical direction (i.e., in the +/−Z direction). In other embodiments, the first lumen 28 may be positioned alongside of the second lumen 30 and extend along the same plane as the second lumen 30. In other embodiments, the second lumen 30 may be positioned above the first lumen 28 in the vertical direction (i.e., in the +/−Z direction). The first lumen 28 and the second lumen 30 may generally take any positionings within the deployment catheter 14.

The first lumen 28 may include the first proximate opening 32a and a distal opening 50 opposite the first proximate opening 32a. The distal opening 50 may be formed at the distal end 22 of the deployment catheter 14 and may be in communication with the deployment portion 42 such that the distal opening 50 couples to the marker receiving opening 44 of the cutting tip 20. As such, the deployment portion 42 and the first lumen 28 form a lumen extending the entire length of the deployment catheter 14 (e.g., extending the entire length of the first lumen 28 and the cutting tip 20 and opening at the marker deployment opening 46 at the beveled surface 34). In some embodiments, the deployment portion 42 of the cutting tip 20 may extend along the same axis as the first lumen 28. In other embodiments, the marker receiving opening 44 of the deployment portion 42 may couple to the distal opening 50 of the first lumen 28, and the deployment portion 42 may be angled or displaced or not positioned along the same axis as the first lumen 28.

Still referring to FIGS. 1-4, the second lumen 30 may include an elongated section 45 extending between the deployment opening 48 and the second proximate opening 32b opposite the deployment opening 48. A ramped portion 52 may be positioned adjacent to or connecting the deployment opening 48 to the first lumen 28 and to the elongated section 45. In some embodiments, the ramped portion 52 extends from the elongated section 45 of the second lumen 30 to the first lumen 28 and connects the deployment opening 48 to the first lumen 28 adjacent to or near or at the distal opening 50 of the first lumen 28. As such, the ramped portion 52 may open into the first lumen 28 and/or into the distal opening 50. The second lumen 30 may be substantially linear until the ramped portion 52, which then adds a slope to the second lumen 30. As such, in some embodiments, a start portion 54 of the ramped portion 52 is positioned opposite (e.g., below) an end portion 56 of the ramped portion 52 with the incline surface 58 connecting the start portion 54 to the end portion 56.

In some embodiments, the incline surface 58 of the ramped portion 52 may extend between the start portion 54 and the end portion 56 and such that the incline surface 58 contiguously couples the distal opening 50 of the first lumen 28 and the deployment opening 48 of the second lumen 30. In some embodiments, the incline surface 58 of the ramped portion 52 may be angled at an obtuse angle. In other embodiments, the incline surface 58 of the ramped portion 52 may be angled at an acute angle extending between the start portion 54 and the end portion 56 such that the incline surface 58 is contiguously couples the distal opening 50 of the first lumen 28 and the deployment opening 48 of the second lumen 30. Further, in some embodiments, the start portion 54, the end portion 56, and the incline surface 58 of the ramped portion 52 are rotated within the deployment catheter 14 such that the first lumen 28 and the second lumen 30 are on the same plane and the incline surface 58 merges the second lumen 30 into the first lumen 28 in the same plane without a change in slope in the device vertical direction (i.e., in the +/−Z direction of the coordinate axes of FIG. 2B), but instead merges the second lumen 30 into the first lumen 28 in the device lateral direction (i.e., in the +/−Y direction of the coordinate axes of FIG. 2B). As such, in this embodiment, the incline surface 58 still contiguously couples the distal opening 50 of the first lumen 28 to the deployment opening 48 of the second lumen 30.

In other embodiments, the portions of the ramped portion 52 (e.g., the start portion 54, the end portion 56, and/or the incline surface 58) may be partially formed in the cutting tip 20, and partially formed in the deployment catheter 14. In other embodiments, the ramped portion 52 (e.g., the start portion 54, the end portion 56, and/or the incline surface 58) may be entirely formed in the cutting tip 20.

It should be understood that because the second lumen 30 is in communication with the first lumen 28 and the first lumen 28 is in communication with the deployment portion 42, this arrangement permits for objects, such as the plurality of markers 18 to pass through the second lumen 30 into the first lumen 28 and then from the first lumen 28 through the marker deployment opening 46, as discussed in greater detail herein. Further, such an arrangement allows for visually determining the number of markers of the plurality of markers 18 by the user, as discussed in greater detail herein. Additionally, such an arrangement permits for individual deployment of a marker of the plurality of markers 18 at a precise desired location, as discussed in greater detail herein.

Still referring to FIGS. 1-4, the first push rod 15 may be positionable within the first lumen 28 of the deployment catheter 14 and the second push rod 16 may be positionable within the second lumen 30 of the deployment catheter 14. The first push rod 15 may include a proximal end 60 and an opposite distal end 62. A tip 64 may be positioned at the distal end 62. An elongated body 66 extends from the distal end 62 to the proximal end 60. The tip 64 may be positioned distal to distal end 62 and may be contoured or shaped to provide increased contact between the tip 64 of the first push rod 15 and the plurality of markers 18, as discussed in greater detail herein. In other embodiments, the tip 64 may be the distal end 62 of first push rod 15.

In some embodiments, the first push rod 15 is illustrated as being generally square in a cross-sectional shape. This is non-limiting and in some embodiments, the first push rod 15 may have a cross-sectional shape that is tubular, rectangular, cylindrical, cone, helix, and/or the like. Further, in some embodiments, the first push rod 15 has a uniform shape between the distal end 62 and the proximal end 60. In other embodiments, the first push rod 15 includes portions that are differently shaped, has a different thickness, a different circumference or diameter, and/or the like.

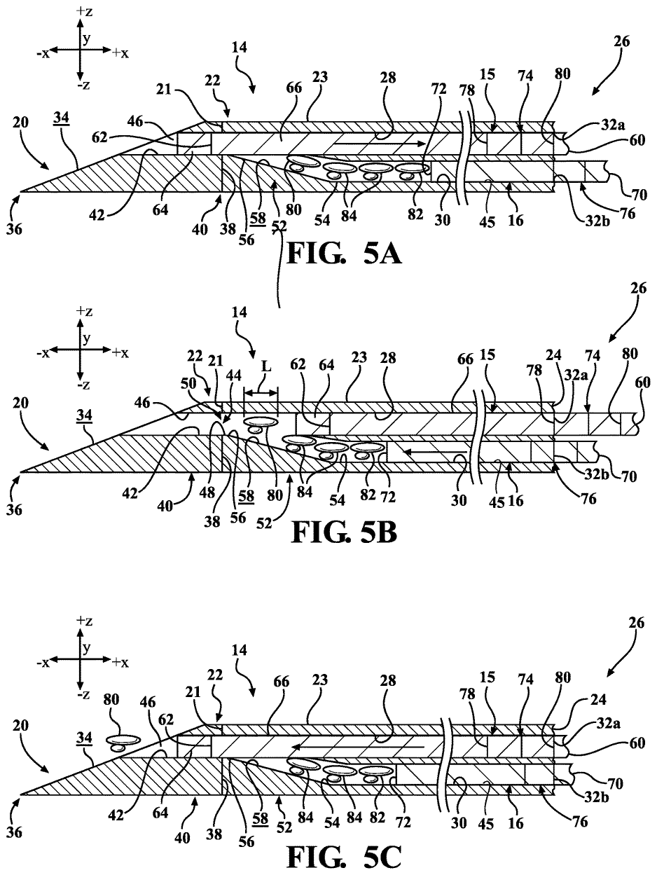

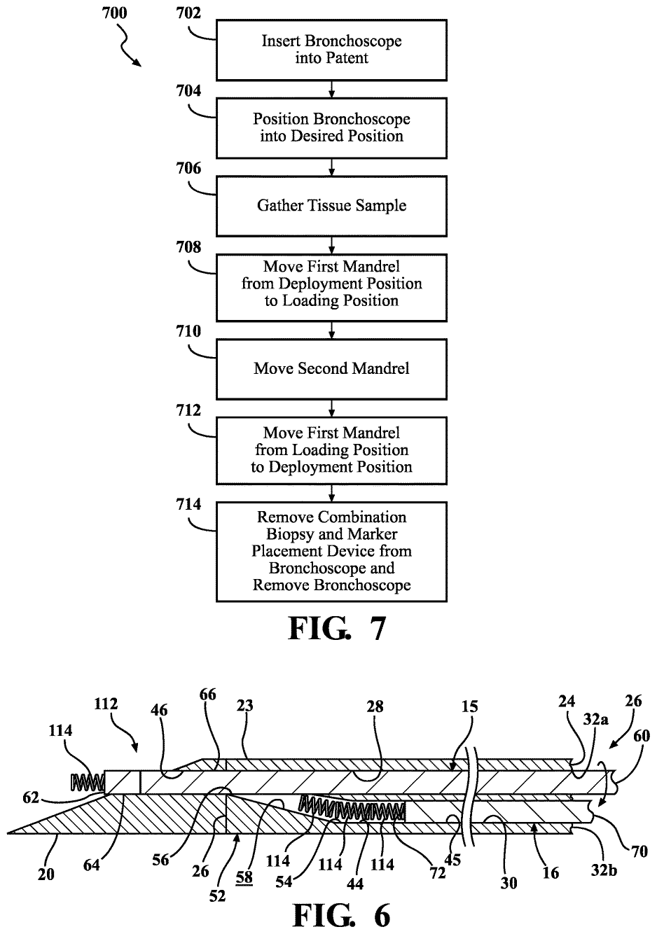

In some embodiments, the tip 64 may include a mechanism for engaging and maintaining contact with one of the plurality of markers 18. The mechanism may be, for example, a magnet. In other embodiments, the mechanism may be, for example, the shape or contour of the tip 64. In other embodiments, the mechanism may be, for example, friction surfaces, adhesive, and/or the like. The distal end 62 may extend through the first proximate opening 32a to be positioned within the first lumen 28. The first push rod 15 may be movable along a length of the first lumen 28 such that the distal end 62 of the first push rod 15 may traverse the first lumen 28 between the first proximate opening 32a and the distal opening 50. The proximal end 60 of the first push rod 15 may extend beyond the opening 24 of the deployment catheter 14 to be physically manipulable by a user, such that the user may move the first push rod 15 along the length of the deployment catheter 14, as discussed in greater detail herein. For example, a first handle 19a of the actuator 19 may be coupled to the proximal end 60 and may be used to manipulate the first push rod 15, as disclosed in greater detail herein. For example, in some embodiments, the first push rod 15 may be manipulable by the user via the first handle 19a of the actuator 19 to move in the device longitudinal direction (i.e., in the +/−X direction of the coordinate axes depicted in FIGS. 5A-5C), as discussed in greater detail herein. Further, in other embodiments, the first push rod 15 may be manipulable by the user via the first handle 19a of the actuator 19 to rotate the first push rod 15, as discussed in greater detail herein with respect to FIG. 6.

The first push rod 15 may be formed of a flexible material, for example, rubber, low density polyethylene, polymethyl methacrylate (PMMA), polycarbonate (PC), polyethylene (PE), polypropylene (PP), polyethylene terephthalate (PETE or PET), polyvinyl chloride (PVC), acrylonitrile-butadiene-styrene (ABS), and/or the like, such that the first push rod 15 may bend to traverse tortuous anatomy of the subject 4. In other embodiments, the first push rod 15 may be formed of a flexible steel or alloy, nylon, and/or the like.

The tip 64 may be formed of a softer material than the first push rod 15, such as nitinol or the like, to prevent damage to the plurality of markers 18 during contact, as discussed in greater detail herein. In other embodiments, the tip 64 may be formed from acrylic or polymethyl methacrylate (PMMA), polycarbonate (PC), polyethylene (PE), polypropylene (PP), polyethylene terephthalate (PETE or PET), polyvinyl chloride (PVC), acrylonitrile-butadiene-styrene (ABS), and/or the like.

In other embodiments, the first push rod 15 and the tip 64 may be formed of the same material. In other embodiments, portions of the first push rod 15 and the tip 64 may be formed of the same material while portions of either or both the first push rod 15 and the tip 64 are formed from different materials.

Still referring to FIG. 1-4, the second push rod 16 may be positioned within the second lumen 30 of the deployment catheter 14. The second push rod 16 may include a proximal end 70 and an opposite distal end 72. An elongated body 68 extends from the distal end 72 to the proximal end 70. The distal end 72 may extend through the second proximate opening 32b to be positioned within the second lumen 30. The distal end 72 of the second push rod 16 may be positioned adjacent the plurality of markers 18 such that the distal end 72 contacts at least one marker of the plurality of markers 18 to advance the markers into the ramped portion 52, as discussed in greater detail herein. The second push rod 16 may be movable along a length of the second lumen 30 such that the distal end 72 of the second push rod 16 may traverse the second lumen 30 between the second proximate opening 32b and the deployment opening 48. The proximal end 70 of the second push rod 16 may extend beyond the opening 24 of the deployment catheter 14. The proximal end 70 of the second push rod 16 may be physically manipulable by a user, such that the user may move the second push rod 16 along the length of the deployment catheter 14, as discussed in greater detail herein. For example, a second handle 19b of the actuator 19 may be coupled to the proximal end 70 and may be used to manipulate the second push rod 16, as disclosed in greater detail herein. For example, the second push rod 16 may be manipulable by the user via the second handle 19b of the actuator 19 to move in the device longitudinal direction (i.e., in the +/−X direction of the coordinate axes depicted in FIGS. 5A-5C), as discussed in greater detail herein.

In some embodiments, the second push rod 16 is illustrated as being generally square in a cross-sectional shape. This is non-limiting and in some embodiments, the second push rod 16 may have a cross-sectional shape that is tubular, rectangular, cylindrical, cone, helix, and/or the like. Further, in some embodiments, the second push rod 16 has a uniform shape between the distal end 72 and the proximal end 70. In other embodiments, the second push rod 16 includes portions that are differently shaped, has a different thickness, a different circumference or diameter, and/or the like. Further, in some embodiments, the shape of the first push rod 15 and the second push rod 16 may be uniform and match one another. In other embodiments, the shape of the first push rod 15 and the second push rod 16 may vary. For example, the first push rod 15 may be a square shape while the second push rod 16 is a cylinder shape.

The second push rod 16 may be formed of a flexible material, for example, rubber, low density polyethylene, polymethyl methacrylate (PMMA), polycarbonate (PC), polyethylene (PE), polypropylene (PP), polyethylene terephthalate (PETE or PET), polyvinyl chloride (PVC), acrylonitrile-butadiene-styrene (ABS), and/or the like, such that the second push rod 16 may bend to traverse tortuous anatomy of the subject 4. In other embodiments, the second push rod 16 may be formed of a flexible steel or alloy, nylon, and/or the like.

Now referring to FIGS. 1, 2A, and 3A, the actuator 19 may be operatively coupled to the proximal end 60 of the first push rod 15 and/or the proximal end 70 of the second push rod 16. The actuator 19 may independently move the first push rod 15 and/or the second push rod 16 along the first lumen 28 and the second lumen 30, respectively, via first and second handles 19a, 19b. The actuator 19 may be a mechanical actuator, such as a handle, a dial, a lever, or the like. The actuator 19 may alternatively be, for example, a linear actuator, a rotary actuator, a hydraulic actuator, a pneumatic actuator, or the like. As depicted, the actuator 19 is a mechanical actuator having the first handle 19a operatively coupled to the proximal end 60 of the first push rod 15 and the second handle 19b operatively coupled to the proximal end 70 of the second push rod 16. Each of the first handle 19a and the second handle 19b may be positioned outside of the first lumen 28 and the second lumen 30 such that the first handle 19a and the second handle 19b may be operated by the user. The first handle 19a may be physically manipulated by the user to move the first push rod 15 along the first lumen 28 between a loading position and a deployment position, as will be described in further detail herein. The second handle 19b may be physically manipulated by the user to move the second push rod 16 along the second lumen 30 to advance the plurality of markers 18 toward the deployment opening 48.

Referring to FIGS. 5A-5C, the combination biopsy and marker deployment device 12 may include a first set of markings 74, each spaced apart by a first predetermined distance formed in the first push rod 15, and a second set of markings 76, each spaced apart by a second predetermined distance formed in the second push rod 16. The first set of markings 74 extend proximally beyond the opening 24 to visually provide the user with a physical representation outside of the first lumen 28 of the position of the first push rod 15 within the first lumen 28. The first predetermined distance may be a distance required for the first push rod 15 to move from a loading position, as best illustrated in FIG. 5B to a deployment position, as best illustrated in FIG. 5C, and described in greater detail herein. As such, as described in greater detail below, the markings may extend proximally beyond the opening 24 or be hidden within the deployment catheter 14 when the first push rod 15 and/or the second push rod 16 are in specific positions within the first lumen 28 and the second lumen 30 respectively, as a visual indicator to the user external to the deployment catheter 14 while portions of the first push rod 15, the second push rod 16, and/or the plurality of markers 18 are positioned within the deployment catheter 14.

In some embodiments, a first marking 78 of the first set of markings 74 may indicate when the first push rod 15 is in the loading position, as best illustrated in FIG. 5B, and a distal marker 80 of the first set of markings 74 may indicate when the first push rod 15 is in the deployment position, as best illustrated in FIG. 5C. Each marking of the first set of markings 74 may indicate the position of the first push rod 15 when the marking is aligned with the proximal end 26 of the deployment catheter 14. The second set of markings 76 may provide the user with a physical representation of the position of second push rod 16. The second predetermined distance may be the predetermined distance required for moving one of the plurality of markers 18 through the deployment opening 48 and into the deployment portion 42 of the first lumen 28 and/or cutting tip 20, as discussed in greater detail herein. The second set of markings 76 may include a number of markings equal to the number of markers of the plurality of markers 18, such that each marking indicates a position of one of the plurality of markers 18 with respect to the deployment opening 48, or the position of the distal end 72 of the second push rod 16 with respect to the deployment opening 48. Similarly to the first set of markings 74, each marking of the second set of markings 76 may indicate the position of the second push rod 16 when the marking is aligned with the proximal end 26 of the deployment catheter 14.

Referring back to FIGS. 1-4 and 5A-5C, the plurality of markers 18 may be stored in the second lumen 30. In some embodiments, when the plurality of markers 18 include at least three markers, there may be a distal marker 80, a proximal marker 82, and at least one intermediate markers 84 positioned between the distal marker 80 and the proximal marker 82. It should be appreciated that as the markers of the plurality of markers 18 are deployed, there may be less than three markers. However, for ease of explanation, the embodiments disclosed herein reference to three or more markers of the plurality of markers 18 stored in the second lumen 30.

In FIG. 5A, the distal marker 80 is illustrated as being stored on the ramped portion 52 of the second lumen 30 near the deployment opening 48. The proximal marker 82 may be positioned in the second lumen 30 before or at the start portion 54 in the device longitudinal direction (i.e., in the +/−X direction of the coordinate axes depicted in FIGS. 5A-5C). The plurality of markers 18 may be positioned in sequence along the second lumen 30, with each marker of the plurality of markers 18 abutting the adjacent markers positioned directly distal and proximal to the marker in the device longitudinal direction (i.e., in the +/−X direction of the coordinate axes depicted in FIGS. 5A-5C). The plurality of markers 18 may be sized and shaped to fit within and be movable along the length of the second lumen 30. The plurality of markers 18 may additionally be sized and shaped to be able to pass through the deployment opening 48 of the second lumen 30.

The plurality of markers 18 may include any number of markers 18, including, for example, one, two, three, four, or more than four. The plurality of markers 18 may be shaped as any known marker capable of marking a location of a biopsy site, such as, for example, a coil, heart, ribbon, hourglass, or the like. Each of the plurality of markers 18 may be screw-shaped that may be rotated into the subject's body at the biopsy site. Each of the markers of the plurality of markers 18 may be formed of, for example, titanium, stainless steel, carbon-coated ceramic, carbon-coated zirconium oxide, nitinol, or the like. As such, each of the plurality of markers 18 may be formed of a material or include a material that is detectable from outside of the body of the subject 4, such as, for example, a radiopaque material.

Referring now to FIGS. 5A-5C, the operation of the combination biopsy and marker deployment device 12 will be described. The first push rod 15 may be movable between the loading position, as best illustrated in FIG. 5B, and the deployment position, as best illustrated in FIGS. 5A and 5C, along the device longitudinal direction (i.e., in the +/−X direction of the coordinate axes depicted in FIGS. 5A-5C). In the loading position, the first push rod 15 may be spaced apart from the deployment portion 42 and the marker receiving opening 44 to permit at least the distal marker 80 of the plurality of markers 18 to move from the second lumen 30 to the deployment opening 48 via the ramped portion 52. With the distal marker 80 positioned in the deployment opening 48, the first push rod 15 may extend through the marker receiving opening 44 and into the deployment portion 42 of the cutting tip 20 to advance the distal marker 80 in the device longitudinal direction (i.e., in the +/−X direction of the coordinate axes depicted in FIGS. 5A-5C) and to close off the deployment opening 48, restricting the ingress and egress of the remaining plurality of markers 18 stored in the second lumen 30 through the deployment opening 48, as best illustrated in FIG. 5C. When moving from the loading position to the deployment position, the first push rod 15 may move one of the markers 18 positioned in the deployment portion 42 of the first lumen 28 and/or cutting tip 20 out of the deployment catheter 14 via the marker deployment opening 46 such that the distal marker 80 is now positioned at a target marking location of the subject 4.

The second push rod 16 may independently push each one of the plurality of markers 18 from the second lumen 30 into the first lumen 28 via the ramped portion 52 and the deployment opening 48. The second push rod 16 may be advanced in the device longitudinal direction (i.e., in the +/−X direction of the coordinate axes depicted in FIGS. 5A-5C) within the second lumen 30 to advance each of the plurality of markers 18 toward the ramped portion 52 and the deployment opening 48. When moving toward the deployment opening 48, the contact between the distal end 72 of the second push rod 16 and the proximal marker 82 may advance each of the plurality of markers 18 toward the deployment opening 48 and positions at least one of the plurality of markers 18 onto the incline surface 58 of the ramped portion 52, as best illustrated in FIG. 5B. The second push rod 16 may advance the markers 18 a predetermined distance required for moving one of the plurality of markers 18 through the deployment opening 48 and into the deployment portion 42 of the first lumen 28/cutting tip 20. The second push rod 16 may bend when the distal end 72 of the second push rod 16 is positioned at the ramped portion 52, such that the second push rod 16 may advance up the ramped portion 52 in the second lumen 30 to advance the proximal marker 82 into the deployment portion 42.

Referring now to FIG. 6, a second illustrative combination biopsy and marker placement device 112 is schematically depicted. It is understood that the combination biopsy and marker placement device 112 is similar to combination biopsy and marker deployment device 12 with the exceptions of the features described herein. As such, like features will use the same reference numerals for the reference characters. As such, for brevity reasons, these features will not be described again.

The combination biopsy and marker placement device 112 may include the deployment catheter 14, the first push rod 15, and the second push rod 16 of the combination biopsy and marker deployment device 12 described above, which will not be described again for brevity reasons. The combination biopsy and marker placement device 112 may include a plurality of markers 114 that, when deployed, are inserted into a portion of the subject 4 at the biopsy site. Each of the plurality of markers 114 may include a receiving helicoid or screw shaped and compliment a shape of the tip 64 of the first push rod 15 such that each of the plurality of markers 114 are configured to be releasably attached or releasably coupled to the tip 64 of the first push rod 15 via a receiving or inverse helicoid. Further, each of the plurality of markers 114 may also be helicoid or screw shaped and configured to be screwed into the subject 4 to mark or otherwise distinguish the desired location on the subject 4. That is, the tip 64 of the first push rod 15 may be configured to rotatably engage with each of the plurality of markers 114 to temporally couple each of the plurality of markers 114 to the tip 64 such that the tip may move the coupled marker into the desired marking location of the subject 4 and then couple the marker to the desired location by rotating the marker of the plurality if markers 114 into the subject 4. As such, the tip 64 may include specific structure such as a straight edge, blade, grooves, threads, and/or the like that engages with the receiving or inverse helicoid of the marker of the plurality of markers 114 to releasably attach the marker of the plurality of markers 114 to the tip 64 and which permits for the marker of the plurality of markers 114 to be rotated into the subject 4 to place the maker at the desired location within the subject 4.

In some embodiments, the rotation of the tip 64 and subsequently the marker of the plurality of markers 114 is by rotating the actuator 19 (e.g., the first handle 19a and the second handle 19b), which may rotate both the first push rod 15 and the second push rod 16 to secure the marker of the plurality of markers 114 to the tip 64 and to place the marker of the plurality of markers 114 at the biopsy site. In some embodiments, the threads may be oppositely wound for different rotation of the actuator 19. For example, the receiving or inverse helicoid of the marker of the plurality of markers 114 may be a left handed thread and the helicoid or screw shape of the marker of the plurality of markers 114 may be a right handed thread and/or vice versa. In other embodiments, the rotation of the tip 64 and subsequently the marker of the plurality of markers 114 is by rotating the first handle 19a only, which rotates only the first push rod 15 to secure the marker of the plurality of markers 114 to the tip 64 and to place the marker of the plurality of markers 114 at the biopsy site. It should be understood that the first and second handles 19a, 19b and the elongated bodies 66, 68 are flexible such that the first handle 19a and elongated body 66 may be separated from the elongated body 68 and/or the second handle 19b to create the space necessary to rotate the first handle 19a.

Each of the plurality of markers 114 may be positioned within the second lumen 30 and moved into the deployment portion 42 by the action of the first and second push rods 15, 16 in a similar manner to the plurality of markers 18 discussed above with respect to FIGS. 1-5.

When positioned in the deployment portion 42, the tip 64 of the first push rod 15 may contact the marker of the plurality of markers 114 to maintain contact with the marker with the tip 64, as discussed above. The first push rod 15 may be extended to the deployment position, with the marker 114 extending out of the first lumen 28 through the marker deployment opening 46. When the marker 114 is in contact with tissue of the subject 4, the first push rod 15 may be rotated, thereby rotating the marker 114 into the subject 4, as discussed above. Once the marker 114 is placed in the tissue, the first push rod 15 may be retracted toward the loading position.

Referring still to FIGS. 1-4, 5A-5C, and 6, and now to FIG. 7, a flowchart of a method 700 of marking a biopsy site with the combination biopsy and marker deployment device 12 is depicted. Although the steps associated with the blocks of FIG. 7 will be described as being separate tasks, in other embodiments, the blocks may be combined or omitted. Further, while the steps associated with the blocks of FIG. 7 will described as being performed in a particular order, in other embodiments, the steps may be performed in a different order.

At block 702, the bronchoscope 2 may be inserted into the subject. The insertion may be via a nose (nasal passageway) or mouth (oral passageway) of the subject and may traverse into the trachea. In the insertion position, in some embodiments, the first push rod 15 may initially be in the deployment position, with the plurality of markers 18 positioned in the second lumen 30 and the deployment opening 48 blocked by the tip 64 of the first push rod 15. In other embodiments, the bronchoscope 2 is first inserted and positioned and then the combination biopsy and marker deployment device 12 is positioned within the bronchoscope 2 with the first push rod 15 initially in the deployment position, with the plurality of markers 18 positioned in the second lumen 30 and the deployment opening 48 blocked by the tip 64 of the first push rod 15.

At block 704, the bronchoscope 2 is positioned at a desired location. The location may be viewed by the user to find the desired location. Once the location is viewed and determined to be a location for the biopsy site, a tissue sample is gathered, at block 706. In some embodiments, the combination biopsy and marker deployment device 12 may be removed from the bronchoscope 2 to retrieve the sample. In other embodiments, the sample is stored on the cutting tip 20. With the first push rod 15 in the deployment position, the distal end 22 of the first push rod 15 may seal off the first lumen 28 to prevent ingress of tissue during removal of the tissue sample.

At block 708, the first push rod 15 may be moved from the deployment position rearward to the loading position in the device longitudinal direction (i.e., the +/−X direction) with respect to the cutting tip 20. Once in the loading position, the deployment opening 48 is open to the first lumen 28 to allow at least one of the plurality of markers 18 to be movable from the second lumen 30 to the first lumen 28 based on the force already applied by the second push rod 16 or will be deployed by the second push rod 16. The first push rod 15 may be moved through actuation of the actuator 19, or through physical manipulation of the first handle 19a. At block 710, the second push rod 16 may be moved along the second lumen 30 in the device longitudinal direction (i.e., the +/−X direction) toward the deployment opening 48, thereby advancing the plurality of markers 18 toward the deployment opening 48. The second push rod 16 may be advanced the predetermined distance equal to a distance required to move the proximal marker 82 of the plurality of markers 18 through the deployment opening 48 to the deployment portion 42. The second push rod 16 may be moved through actuation of the actuator 19, or through physical manipulation of the first handle 19a. The second push rod 16 may advance more than one marker of the plurality of markers 18 at a time. It should be appreciated that in the embodiments of FIG. 6, the first push rod 15 may be rotated to removably attach the marker of the plurality of markers 114 to the tip 64 of the first push rod 15.

At block 712, the first push rod 15 may be moved from the loading position to the deployment position in the device longitudinal direction (i.e., the +/−X direction of the coordinate axes depicted in FIGS. 5A-5C), moving the marker positioned in the deployment portion 42 through the marker deployment opening 46 and deploying the distal marker 80 into the body of the subject 4 at the biopsy site. It should be appreciated that in the embodiments of FIG. 6, the first push rod 15 may be rotated to detach the marker of the plurality of markers 18 from the tip 64 of the first push rod 15 and into the subject 4 at the biopsy site.

The first push rod 15 and the second push rod 16 may be operated to place additional markers at the biopsy site if desired. The combination biopsy and marker deployment device 12 may additionally, or alternatively, be moved to a location of a second biopsy site to remove another tissue sample and place additional markers of the plurality of markers 18. At block 714, when all desired tissue samples are removed, the combination biopsy and marker deployment device 12 may be removed from the channel 6 of the bronchoscope 2 and the bronchoscope 2 may be removed from the subject 4.

Embodiments of the present disclosure may be further described with respect to the following numbered clauses:
1. A device for deploying markers, the device comprising a deployment catheter comprising an elongate body and a cutting tip at a distal end of the elongate body, the deployment catheter defining a first lumen and a second lumen, the first lumen separate from the second lumen, the first lumen having a first proximal opening and a distal opening at the cutting tip, a deployment portion positioned therebetween, the second lumen having a deployment opening and a second proximal opening, the second lumen shaped and sized to hold a plurality of markers, the deployment opening open to the deployment portion of the first lumen; a first push rod positionable within and movable along a length of the first lumen; and a second push rod positionable within and movable along a length of the second lumen independently of the first push rod, the second push rod configured to independently push each one of the plurality of markers from the second lumen into the first lumen via the deployment opening and into a deployed position within the deployment portion, the first push rod configured to push the one of the plurality of markers from the deployed position out of the distal opening at the cutting tip.
2. The device of clause 1, the first push rod is movable between a loading position and a deployment position, wherein in the loading position, the first push rod is spaced apart from the deployment portion and the deployment opening, and in the deployment position, the first push rod extends into the deployment portion to block the deployment opening.
3. The device of any preceding clause, in which the second lumen includes an elongated section and a ramped portion that extends at an angle from the elongated section to intersect the first lumen.
4. The device of any preceding clause, in which the intersection of the elongated section and the ramped portion is at the deployment opening.
5. The device of any preceding clause, wherein the ramped portion and the deployment opening of the second lumen are formed in the elongate body.
6. The device of any preceding clause, in which the first push rod comprises a body; and a tip positioned distal to the body, wherein the tip is configured to maintain contact with one marker of the plurality of markers when the first push rod pushes the one of the plurality of markers from the deployed position out of the distal opening at the cutting tip.
7. The device of any preceding clause, in which the plurality of markers are helicoid-shaped, and the tip of the first push rod is configured to engage with helicoid-shape of the plurality of markers.
8. The device of any preceding clause, in which the first push rod is rotatable to rotate the helicoid-shaped marker of the plurality of markers maintained on the tip.
9. The device of any preceding clause, further comprising a first handle operatively coupled to a proximal end of the first push rod; and a second handle operatively coupled to a proximal end of the second push rod, wherein the first handle is manipulable to move the first push rod along the length of the first lumen, and the second handle is manipulable to move the second push rod along the length of the second lumen.
10. The device of any preceding clause, further comprising a first set of markings formed in the first push rod, wherein the first set of markings provide a visual representation of a position of the first push rod.
11. The device of any preceding clause, further comprising a second set of markings formed in the second push rod, wherein the second set of markings provide a visual representation of a position of the second push rod.
12. A system for performing a biopsy, the system comprising a bronchoscope with a channel formed therein; a device to be inserted into the channel of the bronchoscope, the device comprising: a deployment catheter comprising an elongate body and a cutting tip at a distal end of the elongate body, the elongate body defining a first lumen and a second lumen, the first lumen separate from the second lumen, the first lumen having a first proximate opening and a distal opening at the cutting tip, a deployment portion positioned therebetween, the second lumen having a deployment opening and a second proximate opening, the second lumen shaped and sized to hold a plurality of markers, the deployment opening open to the deployment portion of the first lumen; a first push rod positionable within and movable along a length of the first lumen; and a second push rod positionable within and movable along a length of the second lumen independently of the first push rod, the second push rod configured to independently push each one of the plurality of markers from the second lumen into the first lumen via the deployment opening and into a deployed position within the deployment portion, the first push rod configured to push the one of the plurality of markers from the deployed position out of the distal opening at the cutting tip.
13. The system of clause 12, wherein: the first push rod moves between a loading position and a deployment position, wherein in the loading position, the first push rod is spaced apart from the deployment portion and the deployment opening, and in the deployment position, the first push rod extends into the deployment portion to close off the deployment opening.
14. The system of any preceding clause, in which the second lumen includes an elongated section and a ramped portion that extends obliquely from the elongated section to intersect the first lumen.
15. The system of any preceding clause, in which the intersection of the elongated section and the ramped portion is at the deployment opening.
16. The system of any preceding clause, in which the ramped portion and the deployment opening of the second lumen are formed in the elongate body.
17. The system of any preceding clause, in which the first push rod includes a body and a tip positioned distal to the elongate body, the tip configured to maintain contact with one marker of the plurality of markers when the first push rod pushes the one of the plurality of markers from the deployed position out of the distal opening at the cutting tip.
18. The system of any preceding clause, in which: the plurality of markers are helicoid-shaped, and the tip of the first push rod is configured to engage with helicoid-shape of the plurality of markers.
19. The system of any preceding clause, in which the first push rod is rotatable to rotate the helicoid-shaped marker of the plurality of markers maintained on the tip.
20. The system of any preceding clause, further comprising: a first handle operatively coupled to a proximal end of the first push rod; and a second handle operatively coupled to a proximal end of the second push rod, wherein the first handle is manipulable to move the first push rod along the length of the first lumen, and the second handle is manipulable to move the second push rod along the length of the second lumen.
21. The system of any preceding clause, further comprising: a first set of markings formed in the first push rod, the first set of markings provide a visual representation of a position of the first push rod.
22. The system of any preceding clause, further comprising: a second set of markings formed in the second push rod, the second set of markings provide a visual representation of a position of the second push rod.
23. A method of marking a biopsy site, the method comprising inserting a bronchoscope into an airway of a subject; positioning the bronchoscope at a desired target marking site; retracting a first push rod along a first lumen, via a first handle of a device for deploying markers inserted into the bronchoscope via an instrument port, to move the first push rod into a loading position; advancing a second push rod along a second lumen of the device for deploying markers, via a second handle of the device for deploying markers inserted into the bronchoscope, the advancing of the second push rod moves at least one marker of a plurality of markers stored in the second lumen into the first lumen via a ramped portion coupling the second lumen to the first lumen; and advancing the first push rod along the first lumen from the loading position into a deployment position via the first handle of the device for deploying markers such that a top portion of the first push rod makes contact with the at least one marker of the plurality of markers to advance the at least one marker of the plurality of markers through a deployment portion and exits the device for deploying markers via a deployment opening.
24. The method of clause 19, wherein a proximal end of the first and second push rods each include visual indicators to indicate to a user a remaining number of markers of the plurality of markers stored in the second lumen and the position of the first push rod.
25. The method of any preceding clause, in which: the plurality of markers are helicoid-shaped, and the tip of the first push rod is configured to engage with helicoid-shape of the plurality of markers.
26. The method of any preceding clause, in which the first push rod is rotated to rotate the helicoid-shaped marker of the plurality of markers maintained on the tip.

It should now be understood that embodiments of the present disclosure are directed to devices, systems, and methods for marking a location of a biopsy site with a biopsy device. The biopsy device includes a deployment catheter defining a pair of lumens therethrough, a first push rod positioned in one of the pair of lumens, a second push rod positioned in the other lumen, and a plurality of markers positioned in the second lumen. The second push rod is movable along the second lumen to move one or more of the plurality of markers from the second lumen into the first lumen. The first push rod is movable along the first lumen to move to a loading position where at least one marker of the plurality of markers is moved from the second lumen to the first lumen via contact by the second push rod, and a deployment position where a tip of the first push rod is advanced to make contact with the at least one marker to deploy the at least one marker positioned in the first lumen into the subject.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A device for deploying markers, the device comprising:
a deployment catheter comprising an elongate body and a cutting tip at a distal end of the elongate body, the deployment catheter defining a first lumen and a second lumen, the first lumen separate from the second lumen, the first lumen having a first proximal opening and a distal opening at the cutting tip, a deployment portion positioned therebetween, the second lumen having a deployment opening and a second proximal opening, the second lumen shaped and sized to hold a plurality of markers, the deployment opening open to the deployment portion of the first lumen;
a first push rod positionable within and movable along a length of the first lumen; and
a second push rod positionable within and movable along a length of the second lumen independently of the first push rod, the second push rod configured to independently push each one of the plurality of markers from the second lumen into the first lumen via the deployment opening and into a deployed position within the deployment portion, the first push rod configured to push the one of the plurality of markers from the deployed position out of the distal opening at the cutting tip.

2. The device of claim 1, wherein:
the first push rod is movable between a loading position and a deployment position, wherein in the loading position, the first push rod is spaced apart from the deployment portion and the deployment opening, and in the deployment position, the first push rod extends into the deployment portion to block the deployment opening.

3. The device according to claim 1, wherein the second lumen includes an elongated section and a ramped portion that extends at an angle from the elongated section to intersect the first lumen.

4. The device of claim 3, wherein the intersection of the elongated section and the ramped portion is at the deployment opening.

5. The device of claim 3, wherein the ramped portion and the deployment opening of the second lumen are formed in the elongate body.

6. The device of claim 1, wherein the first push rod comprises:
a body; and
a tip positioned distal to the body,
wherein the tip is configured to maintain contact with one marker of the plurality of markers when the first push rod pushes the one of the plurality of markers from the deployed position out of the distal opening at the cutting tip.

7. The device of claim 1, wherein
the plurality of markers are helicoid-shaped, and
the tip of the first push rod is configured to engage with helicoid-shape of the plurality of markers.

8. The device of claim 7, wherein the first push rod is rotatable to rotate the helicoid-shaped marker of the plurality of markers maintained on the tip.

9. The device of claim 1, further comprising:
a first handle operatively coupled to a proximal end of the first push rod; and
a second handle operatively coupled to a proximal end of the second push rod, wherein:
the first handle is manipulable to move the first push rod along the length of the first lumen, and
the second handle is manipulable to move the second push rod along the length of the second lumen.

10. The device of claim 1, further comprising:
a first set of markings formed in the first push rod,
wherein the first set of markings provide a visual representation of a position of the first push rod.

11. The device of claim 1, further comprising:
a second set of markings formed in the second push rod,
wherein the second set of markings provide a visual representation of a position of the second push rod.

12. A system for performing a biopsy, the system comprising:
a bronchoscope with a channel formed therein;
a device configured to be inserted into the channel of the bronchoscope, the device comprising:
a deployment catheter comprising an elongate body and a cutting tip at a distal end of the elongate body, the elongate body defining a first lumen and a second lumen, the first lumen separate from the second lumen, the first lumen having a first proximate opening and a distal opening at the cutting tip, a deployment portion positioned therebetween, the second lumen having a deployment opening and a second proximate opening, the second lumen shaped and sized to hold a plurality of markers, the deployment opening open to the deployment portion of the first lumen;
a first push rod positionable within and movable along a length of the first lumen; and
a second push rod positionable within and movable along a length of the second lumen independently of the first push rod, the second push rod configured to independently push each one of the plurality of markers from the second lumen into the first lumen via the deployment opening and into a deployed position within the deployment portion, the first push rod configured to push the one of the plurality of markers from the deployed position out of the distal opening at the cutting tip.

13. The system of claim 12, wherein:
the first push rod is movable between a loading position and a deployment position, wherein in the loading position, the first push rod is spaced apart from the deployment portion and the deployment opening, and
in the deployment position, the first push rod extends into the deployment portion to close off the deployment opening.

14. The system according to claim 12, in which the second lumen includes an elongated section and a ramped portion that extends obliquely from the elongated section to intersect the first lumen.

15. The system according to claim 14, wherein the intersection of the elongated section and the ramped portion is at the deployment opening.

16. The system according to claim 14, wherein the ramped portion and the deployment opening of the second lumen are formed in the elongate body.

17. The system according to claim 12, wherein the first push rod includes a body and a tip positioned distal to the elongate body, the tip being configured to maintain contact with one marker of the plurality of markers when the first push rod pushes the one of the plurality of markers from the deployed position out of the distal opening at the cutting tip.

18. The system according to claim 12, wherein
the plurality of markers are helicoid-shaped,
the tip of the first push rod is configured to engage with helicoid-shape of the plurality of markers, and
the first push rod is rotatable to rotate the helicoid-shaped marker of the plurality of markers maintained on the tip.

19.-20. (canceled)

21. The system according to claim 12, further comprising:
a first set of markings formed in the first push rod, the first set of markings provide a visual representation of a position of the first push rod, and
a second set of markings formed in the second push rod, the second set of markings provide a visual representation of a position of the second push rod.

22. (canceled)

23. A method of marking a biopsy site, the method comprising:
inserting a bronchoscope into an airway of a subject;
positioning the bronchoscope at a desired target marking site;
retracting a first push rod along a first lumen, via a first handle of a device for deploying markers inserted into the bronchoscope via an instrument port, to move the first push rod into a loading position;
advancing a second push rod along a second lumen of the device for deploying markers, via a second handle of the device for deploying markers inserted into the bronchoscope, the advancing of the second push rod moves at least one marker of a plurality of markers stored in the second lumen into the first lumen via a ramped portion coupling the second lumen to the first lumen; and
advancing the first push rod along the first lumen from the loading position into a deployment position via the first handle of the device for deploying markers such that a top portion of the first push rod makes contact with the at least one marker of the plurality of markers to advance the at least one marker of the plurality of markers through a deployment portion and exits the device for deploying markers via a deployment opening.

24.-26. (canceled)
